(19) United States
(12) Patent Application Publication
(10) Pub. No.: US 2017/0360389 A1
(43) Pub. Date: Dec. 21, 2017

(54) MEDICAL IMAGE DIAGNOSTIC APPARATUS

(71) Applicant: Toshiba Medical Systems Corporation, Otawara-shi (JP)

(72) Inventors: Rie OCHIAI, Nasushiobara (JP); Yoshimasa KOBAYASHI, Nasushiobara (JP); Naoko KURATOMI, Sakura (JP)

(73) Assignee: Toshiba Medical Systems Corporation, Otawara-shi (JP)

(21) Appl. No.: 15/627,888

(22) Filed: Jun. 20, 2017

(30) Foreign Application Priority Data
Jun. 20, 2016 (JP) 2016-121594

(51) Int. Cl. A61B 6/00; A61B 6/04

(52) U.S. Cl. CPC A61B 6/502; A61B 6/54; A61B 6/4435; A61B 6/0414; A61B 6/5229

(57) ABSTRACT

A medical image diagnostic apparatus according to an embodiment includes a compression plate, an X-ray tube, an arm, an X-ray detector, and an ultrasound probe. The compression plate compresses a breast of a subject. The X-ray tube radiates X-rays. The arm holds the X-ray tube and moves, while the X-ray tube radiating the X-rays, an irradiation region of the X-rays in a direction perpendicular to a front-rear direction of the compression plate. The X-ray detector detects the X-rays having passed through the breast of the subject. The ultrasound probe transmits and receives an ultrasound wave, the ultrasound probe being movable in the direction perpendicular to the front-rear direction of the compression plate.

### CROSS-REFERENCE TO RELATED APPLICATIONS

This application is based upon and claims the benefit of priority from Japanese Patent Application No. 2016-121594, filed on Jun. 20, 2016; the entire contents of which are incorporated herein by reference.

### FIELD

Embodiments described herein relate generally to a medical image diagnostic apparatus.

### BACKGROUND

Conventionally, mammography apparatuses and ultrasound diagnostic apparatuses are used for breast cancer examinations. For example, mammography apparatuses have a high capability of rendering microcalcifications and are capable of imaging the entirety of a breast. In contrast, for example, ultrasound diagnostic apparatuses have a high capability of rendering a tumor and are capable of making a qualitative diagnosis of breasts. As explained herein, mammography apparatuses and ultrasound diagnostic apparatuses have advantages that are different from each other. For breast cancer examinations, it is possible to improve breast cancer detection rates by performing the examinations while using both of the two types of apparatuses.

As an example of the mammography apparatuses described above, an apparatus is known in which a photon-counting-type detectors are used. For example, in such a photon-counting-type mammography apparatus, detectors are arranged in a single line or a plurality of lines, so that an X-ray image is taken while the detectors move in conjunction with a tubus (which may be called “cone”) serving as a beam limiting cone for the X-rays. By using this photon counting technique, it is possible to obtain three-dimensional information, and it is also possible to identify substances by acquiring data while using a plurality of energy bins.

Further, as an example of the ultrasound diagnostic apparatuses described above, a system called an Automated Breast Ultrasound System (ABUS) is known by which an ultrasound probe automatically scans an examined subject. The ABUS is configured to acquire an ultrasound image by transmitting and receiving an ultrasound wave while the ultrasound probe is automatically sliding. With this arrangement, it is possible to accurately and clearly render a tissue, without depending on skills of medical providers.

### BRIEF DESCRIPTION OF THE DRAWINGS

FIG. 1 is a diagram illustrating an exemplary configuration of a mammography apparatus according to a first embodiment;

FIG. 2 is a drawing for explaining operations of functional units of the mammography apparatus according to the first embodiment;

FIG. 3 is a drawing for explaining operations of an X-ray tube and an X-ray detector according to the first embodiment;

FIG. 4 is a drawing for explaining control related to acquiring ultrasound images and X-ray images according to the first embodiment;

FIG. 5 is a drawing for explaining examples of moving distances of the X-ray detector and an ultrasound probe according to the first embodiment;

FIG. 6A is a drawing illustrating an example of an ultrasound image according to the first embodiment;

FIG. 6B is a chart illustrating an example of an X-ray radiation condition according to the first embodiment;

FIG. 7 is a drawing illustrating an example of X-ray radiation control according to the first embodiment;

FIG. 8 is a flowchart illustrating a procedure in a process performed by the mammography apparatus according to the first embodiment; and

FIG. 9 is a drawing illustrating examples of a compression plate and a Bucky's device according to a second embodiment.

### DETAILED DESCRIPTION

According to an embodiment, a medical image diagnostic apparatus includes a compression plate, an X-ray tube, an arm, an X-ray detector and an ultrasound probe. The compression plate is configured to compress a breast of a subject. The X-ray tube is configured to radiate X-rays. The arm is configured to hold the X-ray tube and to move, while the X-ray tube radiating the X-rays, an irradiation region of the X-rays in a direction perpendicular to a front-rear direction of the compression plate. The X-ray detector is configured to detect the X-rays having passed through the breast of the subject. The ultrasound probe is configured to transmit and receive an ultrasound wave, the ultrasound probe being movable in the direction perpendicular to the front-rear direction of the compression plate.

### First Embodiment

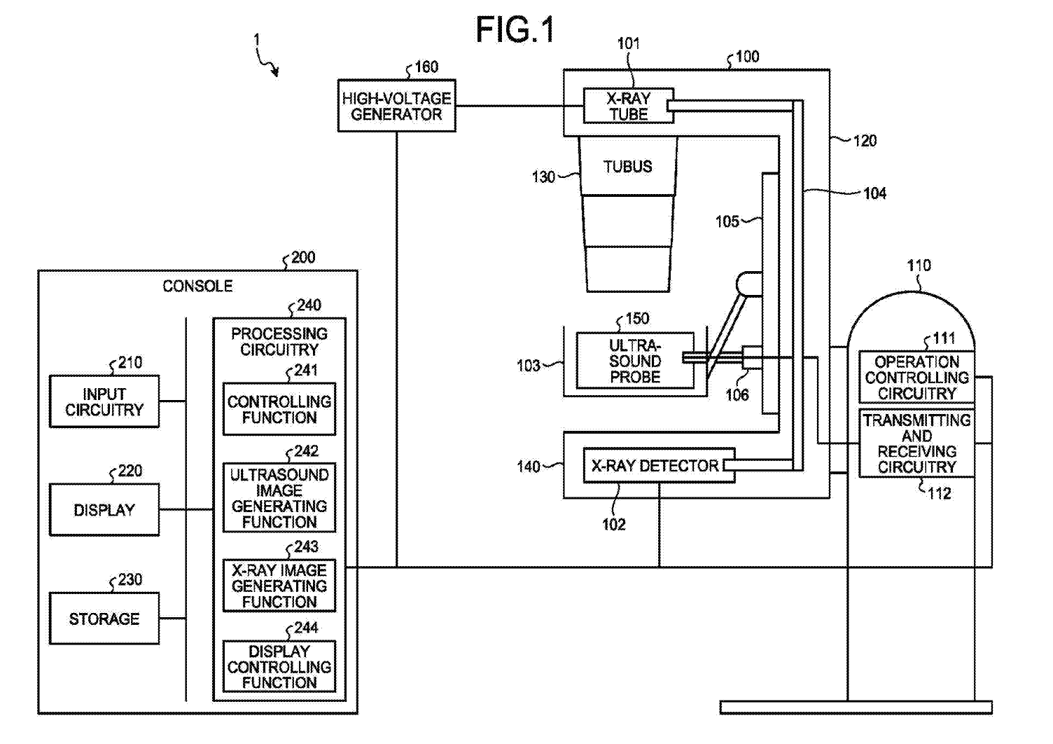

Exemplary embodiments of a medical image diagnostic apparatus of the present disclosure will be explained below, with reference to the accompanying drawings. In the following sections, a mammography apparatus serving as a medical image diagnostic apparatus of the present disclosure will be explained. FIG. 1 is a diagram illustrating an exemplary configuration of a mammography apparatus 1 according to a first embodiment.

As illustrated in FIG. 1, the mammography apparatus 1 according to the first embodiment includes an image taking device 100, a high-voltage generator 160, and a console 200. As illustrated in FIG. 1, the image taking device 100 includes a base unit 110, an arm unit 120, a tubus (which may be called “cone”) 130, a Bucky's device 140, and an ultrasound probe 150. Further, the image taking device 100 includes an X-ray tube 101, an X-ray detector 102, a compression plate 103, a supporting unit 104, upper and lower rails 105, left and right rails 106, operation controlling circuitry 111, and transmitting and receiving circuitry 112.

The base unit 110 is configured to support the arm unit 120 so that the arm unit 120 is able to make up-and-down movements along a vertical direction. Further, the base unit 110 supports the arm unit 120 so that the arm unit 120 is rotatable on an axis extending along a horizontal direction. The arm unit 120 holds the X-ray tube 101 and the X-ray detector 102 so as to oppose each other. Further, the arm unit 120 holds the tubus (a beam limiting cone) 130 used for adjusting a radiation range of X-rays radiated from the X-ray tube 101. Further, the arm unit 120 holds the Bucky's device 140 used for storing therein the X-ray detector 102. Further, by holding the compression plate 103 via the upper and lower rails 105, the arm unit 120 holds the compression plate 103 so that the compression plate 103 is able to advance toward and retreat from the X-ray detector 102. Further, by holding the ultrasound probe 150 via the left and right rails 106, the arm unit 120 holds the ultrasound probe 150 so that the ultrasound probe 150 is movable along a direction orthogonal to the depth direction (i.e., the left-and-right direction in the drawing) of the compression plate 103.

The tubus 130 is structured so as to be able to expand and contract in a direction connecting together the X-ray tube 101 and the X-ray detector 102. Between the X-ray tube 101 and the X-ray detector 102 opposing each other, the tubus 130 is held on the X-ray tube 101 side. The tubus 130 is configured to inhibit the X-rays radiated from the X-ray tube 101 from spreading, so as to form a fan-shaped X-ray beam. Further, the tubus 130 includes a collimator (not illustrated) and is configured to adjust the radiation range of the X-rays radiated from the X-ray tube 101. Further, in conjunction with a rotation of the X-ray tube 101, the orientation of the tip end of the tubus 130 can be varied along a direction orthogonal to the depth direction of the compression plate 103. The varying of the orientation will be explained in detail later. The Bucky's device 140 stores the X-ray detector 102 therein and is configured to have an imaged object (a breast) placed thereon. Further, the Bucky's device 140 holds an X-ray grid configured to improve image contrast by eliminating scattered rays. The Bucky's device 140 is configured to cause the X-ray grid to swing along a direction orthogonal to the orientation of a foil.

The X-ray tube 101 is configured to generate the X-rays on the basis of a voltage applied thereto from a high-voltage generator 160. Further, the X-ray tube 101 is configured to vary the radiation direction of the X-rays by rotating on an axis extending in the horizontal direction. The varying of the radiation direction of the X-rays will be explained in detail later. The X-ray detector 102 is configured to detect X-rays that are radiated by the X-ray tube 101 and have passed through the imaged object. In this situation, in conjunction with the rotational movement of the X-ray tube 101, the position of the X-ray detector 102 is varied along a direction orthogonal to the depth direction of the compression plate 103. The varying of the position will be explained in detail later.

For example, the X-ray detector 102 is a photon counting detector and is configured to detect each of the photons of X-rays that have become incident to each pixel and to count and output the quantity of the photons. In other words, the X-ray detector 102 is configured to output, every time an X-ray photon becomes incident, a signal that makes it possible to measure an energy value of the X-ray photon. In this situation, each of the X-ray photons is, for example, an X-ray photon that was radiated from the X-ray tube 101 and has passed through the imaged object.

The X-ray detector 102 includes a plurality of detecting elements each of which is configured to output a one-pulse electrical signal (an analog signal) every time an X-ray photon becomes incident thereto. The mammography apparatus 1 is capable of counting the quantity of the X-ray photons that have become incident to each of the detecting elements, by counting the quantity of the electrical signals (the pulses). Further, by performing a computation process on these signals, the mammography apparatus 1 is capable of measuring an energy value of the X-ray photons that caused the outputs of the signals.

Each of the detecting elements described above is structured, for example, by using a scintillator and an optical sensor configured with a photomultiplier or the like. In the present example, the X-ray detector 102 is an indirect-conversion type detector configured to convert the X-ray photons that have become incident into scintillator light by using the scintillators and to further convert the scintillator light into the electrical signals by using the optical sensors configured with the photomultipliers or the like. Alternatively, for example, each of the detecting elements described above may be a semiconductor element configured by using cadmium telluride (CdTe) or cadmium zinc telluride (CdZnTe). In that situation, the X-ray detector 102 is a direct-conversion type detector configured to directly convert the X-ray photons that have become incident into electrical signals.

For example, the X-ray detector 102 may be a single-line detector in which the detecting elements are arranged in N rows along the depth direction (the left-and-right direction in the drawing) of the compression plate 103. Alternatively, the X-ray detector 102 may be a multiple-line detector in which the detecting elements are arranged in N rows along the depth direction of the compression plate 103 and in M rows along the direction orthogonal to the depth direction. The compression plate 103 is a compressing tool used for compressing a breast of a patient during an image taking process. The compression plate 103 is connected to the upper and lower rails 105 and is configured to advance toward and retreat from the X-ray detector 102. The two ends of the supporting unit 104 are configured to support the X-ray tube 101 and the X-ray detector 102, respectively. Further, the supporting unit 104 is configured to make a rotational movement realized by a driving force of a motor (not illustrated). The rotational movement will be explained in detail later.

The ultrasound probe 150 is arranged on the inside of the compression plate 103, is connected to the left and right rails 106, and is configured to move along the direction orthogonal to the depth direction of the compression plate 103. For example, the ultrasound probe 150 includes a plurality of piezoelectric transducer elements. The plurality of piezoelectric transducer elements are configured to generate an ultrasound wave on the basis of a drive signal supplied thereto from the transmitting and receiving circuitry 112 (explained later). Further, the ultrasound probe 150 is configured to receive reflected waves from the imaged object and to convert the received reflected waves into electrical signals. Further, the ultrasound probe 150 includes matching layers provided for the piezoelectric transducer elements, as well as a backing member or the like that prevents ultrasound waves from propagating rearward from the piezoelectric transducer elements. In this situation, the ultrasound probe 150 is detachably connected to the left and right rails 106. In other words, the ultrasound probe 150 may be attached and detached in accordance with the status of use. It is therefore possible to use any of various types of ultrasound probes. The ultrasound probe 150 may be a one-dimensional (1D) array probe in which the plurality of piezoelectric transducer elements are arranged in a row or may be a two-dimensional (2D) array probe in which the plurality of piezoelectric transducer elements are arranged in a matrix formation.

The operation controlling circuitry 111 is configured to control the up-and-down movements and the rotational movement of the arm unit 120 and the advancing and retreating movements of the compression plate 103, on the basis of instructions transferred thereto from the console 200. Further, the operation controlling circuitry 111 is configured to control operations of the X-ray tube 101, the X-ray detector 102, the tubus 130, and the ultrasound probe 150, on the basis of instructions transferred thereto from the console 200. Further, the operation controlling circuitry 111 is configured to control the expansion and contraction of the tubus 130 and operations of limiting blades included in the collimator, on the basis of instructions transferred thereto from the console 200.

The transmitting and receiving circuitry 112 includes a pulse generator, a transmission delay unit, a pulser, and the like and is configured to supply the drive signal to the ultrasound probe 150. The pulse generator is configured to repeatedly generate a rate pulse used for forming a transmission ultrasound wave, at a predetermined rate frequency. It is sufficient when the predetermined rate frequency is fixed at the point in time when the ultrasound wave is transmitted and received and may be determined any time prior to that point in time. Further, the transmission delay unit is configured to apply a delay period that is required to converge the ultrasound wave generated by the ultrasound probe 150 into the form of a beam and to determine transmission directionality and that corresponds to each of the piezoelectric transducer elements, to each of the rate pulses generated by the pulse generator. Further, the pulser is configured to apply the drive signal (a drive pulse) to the ultrasound probe 150 with timing based on the rate pulses. In other words, by varying the delay periods applied to the rate pulses, the transmission delay unit arbitrarily adjusts the transmission directions of the ultrasound waves transmitted from the surfaces of the piezoelectric transducer elements.

Further, the transmitting and receiving circuitry 112 includes a pre-amplifier, an Analog/Digital (A/D) converter, a reception delay unit, an adder, and the like and is configured to generate reflected-wave data by performing various types of processes on reflected-wave signals received by the ultrasound probe 150. The pre-amplifier is configured to amplify the reflected-wave signals in correspondence with channels. The A/D converter is configured to perform an A/D conversion on the amplified reflected-wave signals. The reception delay unit is configured to apply a delay period that is required to determine reception directionality. The adder is configured to generate the reflected-wave data by performing an adding process on the reflected-wave signals processed by the reception delay unit. As a result of the adding process performed by the adder, reflected components from the direction corresponding to the reception directionality of the reflected-wave signals are emphasized, so that a comprehensive beam for transmitting and receiving the ultrasound wave is formed on the basis of the reception directionality and the transmission directionality.

The high-voltage generator 160 is configured to generate the high voltage and supplies the generated high voltage to the X-ray tube 101, under control of the console 200 (explained later).

As illustrated in FIG. 1, the console 200 includes input circuitry 210, a display 220, a storage 230, and processing circuitry 240.

The input circuitry 210 is realized with a trackball, a switch button, a mouse, a keyboard, and/or the like, used for establishing various types of settings. The input circuitry 210 is connected to the processing circuitry 240 and is configured to convert an input operation received from an operator into an electrical signal and to output the electrical signal to the processing circuitry 240. The display 220 is configured to display a Graphical User Interface (GUI) used for receiving instructions from the operator and various types of images generated by the processing circuitry 240.

The storage 230 is configured to receive and store therein image data generated by the processing circuitry 240. Further, the storage 230 stores therein an X-ray radiation condition. The radiation condition will be explained in detail later. In addition, the storage 230 stores therein computer programs (hereinafter, “programs”) corresponding to various types of functions read and executed by the circuits illustrated in FIG. 1. In one example, the storage 230 stores therein a program corresponding to a controlling function 241, a program corresponding to an ultrasound image generating function 242, a program corresponding to an X-ray image generating function 243, and a program corresponding to a display controlling function 244 that are read and executed by the processing circuitry 240. Further, the storage 230 stores therein a program corresponding to an operation controlling function and being read and executed by the operation controlling circuitry 111; and a program corresponding to a transmitting and receiving function and being read and executed by the transmitting and receiving circuitry 112.

The processing circuitry 240 is configured to control operations of the entirety of the mammography apparatus 1. More specifically, the processing circuitry 240 performs various types of processes by reading and executing, from the storage 230, the program corresponding to the controlling function 241 configured to control the entire apparatus. For example, the processing circuitry 240 controls the dose of the X-rays radiated onto the imaged object and turns the radiation on/off, by controlling the high-voltage generator 160 and adjusting the voltage supplied to the X-ray tube 101 according to an instruction of the operator transferred thereto from the input circuitry 210. Further, for example, the processing circuitry 240 adjusts the up-and-down movements and the rotational movement of the arm unit 120 and the advancing and retreating movements of the compression plate 103, by controlling the operation controlling circuitry 111 according to an instruction of the operator. Further, the processing circuitry 240 adjusts operations of the X-ray tube 101, the X-ray detector 102, the tubus 130, and the ultrasound probe 150, by controlling the operation controlling circuitry 111 according to an instruction of the operator. Furthermore, for example, the processing circuitry 240 controls the radiation range of the X-rays radiated onto the imaged object, by controlling the operation controlling circuitry 111 and adjusting the expansion and contraction of the tubus 130 and the opening degree of the limiting blades included in the collimator, according to an instruction of the operator.

Further, the processing circuitry 240 is configured to control the transmitting and receiving circuitry 112 according to an instruction of the operator. Further, the processing circuitry 240 is configured to control generating processes of X-ray images and ultrasound images, as well as image processing processes and analyzing processes performed on generated images. In addition, the processing circuitry 240 is configured to exercise control so that the display 220 displays the GUI used for receiving instructions from the operator and any of the images stored in the storage 230.

For example, by reading and executing the program corresponding to the ultrasound image generating function 242 from the storage 230, the processing circuitry 240 is configured to generate various types of ultrasound images. In one example, the processing circuitry 240 generates an ultrasound image from the reflected-wave data generated by the transmitting and receiving circuitry 112. Further, by reading and executing the program corresponding to the X-ray image generating function 243 from the storage 230, the processing circuitry 240 is configured to generate various types of X-ray images. In one example, the processing circuitry 240 generates X-ray image data by using the electrical signals converted from the X-rays by the X-ray detector 102. Further, by reading and executing the program corresponding to the display controlling function 244 from the storage 230, the processing circuitry 240 is configured to cause the display 220 to display any of the ultrasound images and the X-ray images.

A configuration of the mammography apparatus 1 according to the first embodiment has thus been explained. The mammography apparatus 1 according to an embodiment of the present disclosure configured as described above makes it possible to improve the efficiency in image interpretation. More specifically, the mammography apparatus 1 improves the efficiency in image interpretation by making it easier to compare images and shortening the time period required by medical examinations, by eliminating differences in the posture of the examined subject (hereinafter, “patient”) among the images (differences in the state of the patient during the image taking process) by acquiring ultrasound images in a parallel manner from the patient who is undergoing an X-ray image taking process.

As explained above, for breast cancer examinations, it is possible to improve breast cancer detection rates by performing the examinations while using both a mammography apparatus and an ultrasound diagnostic apparatus. However, the level of precision is degraded when the positions of the lesions in the two types of images are compared with each other due to the difference in the posture of the patient during the examinations. (Mammography apparatuses are configured to take images while the breast is compressed, whereas ultrasound diagnostic apparatuses are configured to scan the patient while the patient is lying supine. The shapes of the breast are therefore different between these two examination processes.) To cope with this situation, the mammography apparatus 1 according to an embodiment of the present disclosure improves the efficiency in image interpretation by arranging the shapes of the breast to be the same when acquiring the two types of images, by acquiring ultrasound images in a parallel manner from a patient who is undergoing an X-ray image taking process. Further, the mammography apparatus 1 according to an embodiment of the present disclosure is capable of acquiring X-ray images and ultrasound images at the same time. It is therefore possible to reduce pains of patients taking breast cancer examinations (by exposing their breasts twice) and to shorten the time period required by the examinations.

The compression plate 103 illustrated in FIG. 1 is an example of the compression plate set forth in the claims. Further, the X-ray tube 101 and the tubus 130 illustrated in FIG. 1 are examples of an X-ray radiating unit. The X-ray tube 101 is an example of the X-ray tube set forth in the claims. Further, the X-ray detector 102 illustrated in FIG. 1 is an example of the X-ray detector set forth in the claims. Also, the ultrasound probe 150 illustrated in FIG. 1 is an example of the ultrasound probe set forth in the claims. In addition, the controlling function 241 illustrated in FIG. 1 is an example of the controlling unit set forth in the claims. The controlling unit described in the present disclosure may be realized with a combination of hardware such as a circuit and software. Further, the ultrasound image generating function 242, the X-ray image generating function 243, and the display controlling function 244 illustrated in FIG. 1 are each an example of an ultrasound image generating unit, an X-ray image generating unit, and a display controlling unit, respectively. The ultrasound image generating unit, the X-ray image generating unit, and the display controlling unit may each be realized with a combination of hardware such as a circuit and software.

In the mammography apparatus 1 according to an embodiment of the present disclosure, the X-ray radiating unit is configured to radiate X-rays while moving a radiation region of the X-rays along a direction orthogonal to the depth direction of the compression plate; the X-ray detector is configured to move along a direction orthogonal to the depth direction of the compression plate in conjunction with the moving of the radiation region of the X-rays realized by the X-ray radiating unit; and the ultrasound probe is configured to transmit and receive an ultrasound wave while being moved along a direction orthogonal to the depth direction of the compression plate.

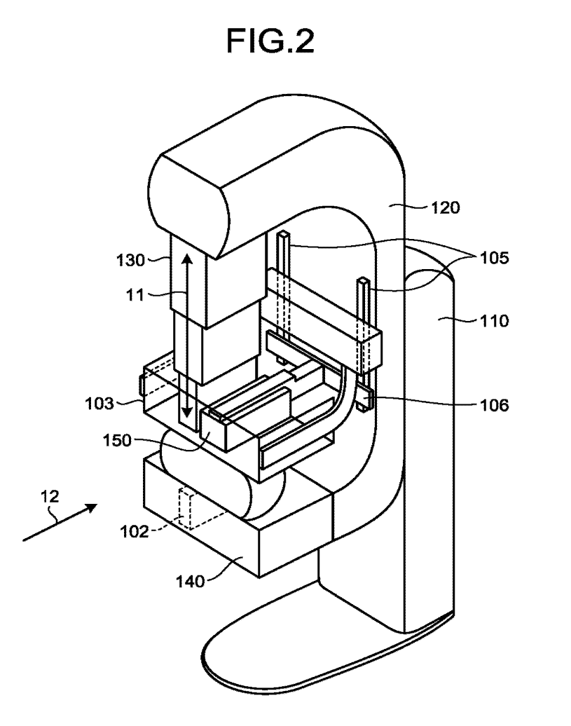

FIG. 2 is a drawing for explaining operations of functional units of the mammography apparatus 1 according to the first embodiment. As illustrated in FIG. 2, in the mammography apparatus 1, the ultrasound probe 150 is connected to the left and right rails 106 provided for the arm unit 120 and is configured to slide and move in the left-and-right direction of the mammography apparatus 1 along the left and right rails 106. In this situation, as illustrated in FIG. 2, the ultrasound probe 150 is structured so that the depth direction of the compression plate 103 corresponds to the lengthwise (the longer side) direction thereof. In other words, the ultrasound probe 150 is configured so as to slide and move along the widthwise (the shorter side) direction thereof while the plurality of piezoelectric transducer elements are arranged along the depth direction of the compression plate 103 (or in the direction orthogonal to the alignment direction of the piezoelectric transducer elements, when the piezoelectric transducer elements are arranged one-dimensionally).

Further, the ultrasound probe 150 and the compression plate 103 are connected to the upper and lower rails 105 provided for the arm unit 120 and are configured to slide and move along the up-and-down direction of the mammography apparatus 1 along the upper and lower rails 105. For example, as illustrated in FIG. 2, the ultrasound probe 150 is configured to slide and move along the up-and-down direction and the left-and-right direction of the mammography apparatus 1 by being connected to the left and right rails 106 connected to the upper and lower rails 105.

Further, as illustrated in FIG. 2, the mammography apparatus 1 includes the tubus 130 positioned on the X-ray tube 101 side of the arm unit 120. The tubus 130 is structured so as to be able to expand and contract in the direction indicated by an arrow 11 in FIG. 2. In the present example, similarly to the ultrasound probe 150, the tubus 130 is structured so that the dimension thereof in the depth direction of the compression plate 103 is longer than the dimension thereof in the direction orthogonal thereto. Further, the tubus 130 is configured so that the tip end thereof is swingable (rotatable) in the left-and-right direction of the mammography apparatus 1, while using a connection part with the arm unit 120 as a point of support.

Further, in the mammography apparatus 1, similarly to the ultrasound probe 150, the X-ray detector 102 is structured so that the dimension thereof in the depth direction of the compression plate 103 is longer than the dimension thereof in the direction orthogonal thereto and is configured to be moved in the direction orthogonal to the depth direction (i.e., the left-and-right direction of the mammography apparatus 1). In other words, while the plurality of detecting elements are arranged along the depth direction of the compression plate 103, the X-ray detector 102 is configured so as to move in the widthwise direction (or in the direction orthogonal to the alignment direction of the detecting elements, when the detecting elements are arranged one-dimensionally). In this situation, the X-ray detector 102 moves in the left-and-right direction, in conjunction with the varying of the radiation direction of the X-rays radiated by the X-ray tube 101.

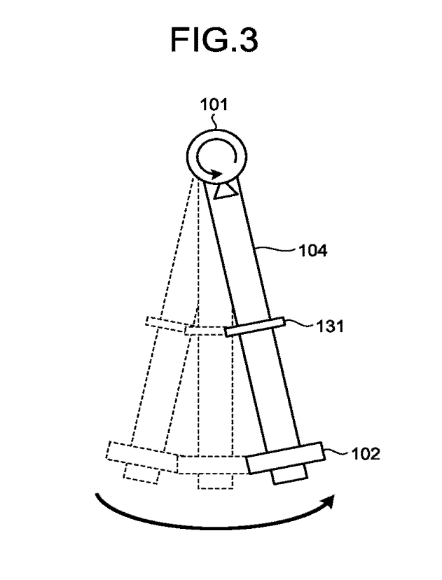

FIG. 3 is a drawing for explaining operations of the X-ray tube 101 and the X-ray detector 102 according to the first embodiment. FIG. 3 illustrates the operations of the X-ray tube 101 and the X-ray detector 102 while the mammography apparatus 1 is viewed in the direction of an arrow 12 in FIG. 2. The X-ray tube 101 and the X-ray detector 102 according to the first embodiment are provided so as to be movable in the mammography apparatus 1. For example, as illustrated in FIG. 3, the X-ray tube 101 is provided so as to rotate on an axis extending in the depth direction of the mammography apparatus 1 (the depth direction of the compression plate 103). In other words, the X-ray tube 101 rotates so as to vary the X-ray radiation direction along the left-and-right direction of the mammography apparatus 1.

Further, for example, as illustrated in FIG. 3, the X-ray detector 102 is configured to move along the left-and-right direction of the mammography apparatus 1, while being supported by the supporting unit 104. For example, as a result of the supporting unit 104 rotating while using a connection part of the X-ray tube 101 as a point of support, the X-ray detector 102 moves along the left-and-right direction. In this situation, the X-ray tube 101 and the X-ray detector 102 are controlled so as to move in conjunction with each other. In other words, the rotation of the X-ray tube 101 and the moving of the X-ray detector 102 are controlled in such a manner that the X-rays radiated from the X-ray tube 101 are detected at all times by the X-ray detector 102 (e.g., in such a manner that the radiation axis of the X-rays go through the center of the X-ray detector 102). Further, the tubus 130 also moves in conjunction with the X-ray tube 101 and the X-ray detector 102. In other words, the tip end of the tubus 130 is rotated along the left-and-right direction of the mammography apparatus 1, while using the connection part with the arm unit 120 as a point of support, in accordance with the radiation direction of the X-rays radiated from the X-ray tube 101. As a result, as illustrated in FIG. 3, a collimator 131 included in the tubus 130 makes a movement that is in conjunction with the X-ray tube 101 and the X-ray detector 102.

The operations illustrated in FIG. 3 are merely examples. As long as the X-rays are radiated toward the X-ray detector 102, any type of operation control may be exercised. For example, it is also acceptable to move the radiation region of the X-rays, as a result of the supporting unit 104 moving in conjunction with the moving of the X-ray detector 102, the supporting unit 104 being configured to support the X-ray tube 101. To describe one example with reference to FIG. 3, for instance, it is acceptable to move the radiation region of the X-rays, as a result of exercising control so that the position of the X-ray tube 101 varies in conjunction with moving of the X-ray detector 102. Further, the rotation direction of the X-ray tube 101 and the moving directions of the X-ray detector 102, the tubus 130, and the ultrasound probe 150 are determined arbitrarily. For example, control may be exercised so as to move the radiation region of the X-rays and the ultrasound probe 150, from the central axis of the body of the patient toward the outside of the body. In one example, control may be exercised so that, at first, the radiation region of the X-rays and the ultrasound probe 150 are positioned near the central axis in the chest of the patient and are subsequently moved from the initial position in the direction toward either the left arm or the right arm of the patient.

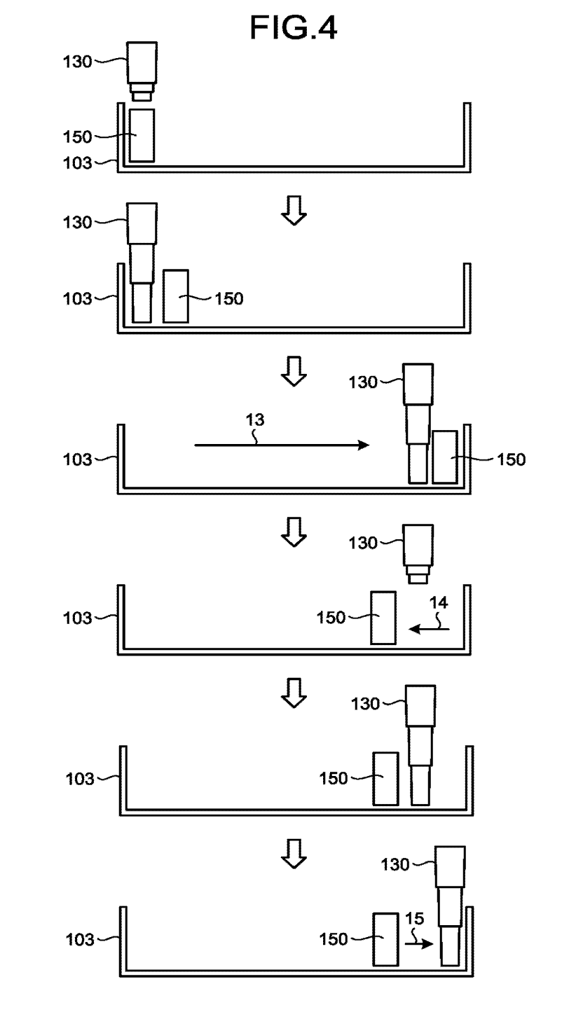

The operations of the functional units have thus been explained. In this situation, the operations of the functional units described above are controlled by the operation controlling circuitry 111. Next, control exercised to acquire X-ray images and ultrasound images will be explained. FIG. 4 is a drawing for explaining the control related to the acquiring of the ultrasound images and the X-ray images according to the first embodiment. FIG. 4 illustrates an example in which the mammography apparatus 1 is viewed in the direction of the arrow 12 in FIG. 2. In this situation, by the mammography apparatus 1 according to the first embodiment, an ultrasound image is acquired before an X-ray image is acquired. More specifically, the ultrasound probe 150 transmits and receives an ultrasound wave to and from the breast of the patient, prior to the radiating of the X-rays onto the breast of the patient performed by the X-ray radiating unit.

For example, as illustrated in the first section of FIG. 4, the operation controlling circuitry 111 moves the tubus 130 to a first end (e.g., the left end) of the two ends of the compression plate 103 and also puts a space between the tubus 130 and the compression plate 103 by causing the tubus 130 to contract. Further, the operation controlling circuitry 111 arranges the ultrasound probe 150 to be in the space. Next, the transmitting and receiving circuitry 112 starts an ultrasound wave transmitting and receiving process, and also, the operation controlling circuitry 111 causes the ultrasound probe 150 to slide and move toward a second end (e.g., the right end).

When a space is made between the tubus 130 and the compression plate 103 as a result of the moving of the ultrasound probe 150, the operation controlling circuitry 111 causes the tubus 130 to expand as illustrated in the second section of FIG. 4, so that the X-ray tube 101 starts radiating X-rays. Further, the operation controlling circuitry 111 moves the ultrasound probe 150 and the tubus 130 in the direction indicated by an arrow 13, as illustrated in the third section of FIG. 4. At this time, the controlling function 241 controls the high-voltage generator 160 and the transmitting and receiving circuitry 112, so as to sequentially acquire X-ray images and ultrasound images. For example, the controlling function 241 exercises control so that an X-ray image and an ultrasound image are acquired, every time the ultrasound probe 150 and the X-ray detector 102 are moved by the operation controlling circuitry 111 by a predetermined distance.

Further, when the ultrasound probe 150 has reached the second end, the operation controlling circuitry 111 puts a space between the tubus 130 and the compression plate 103 by causing the tubus 130 to temporarily contract, as illustrated in the fourth section of FIG. 4 and further moves the ultrasound probe 150 in the opposite direction (in the direction indicated by an arrow 14 in FIG. 4) of the moving direction so far. After that, the operation controlling circuitry 111 causes the tubus 130 to expand as illustrated in the fifth section of FIG. 4 and further moves the tubus 130 in the direction indicated by an arrow 15 (toward the second end), as illustrated in the sixth section of FIG. 4.

As explained above, the mammography apparatus 1 according to the first embodiment is configured to acquire an ultrasound image before acquiring an X-ray image. In this situation, the image acquiring processes are controlled so as to acquire data of the entire imaged object. As explained above, the ultrasound images and the X-ray images are acquired while the functional units are being moved with respect to the imaged object. Accordingly, when acquiring the images, if the ultrasound probe 150 and the X-ray detector 102 were moved by a long distance, there would be a gap between a piece of data acquired before the moving and a piece of data acquired after the moving, and the imaged object would therefore have a region from which no image is acquired.

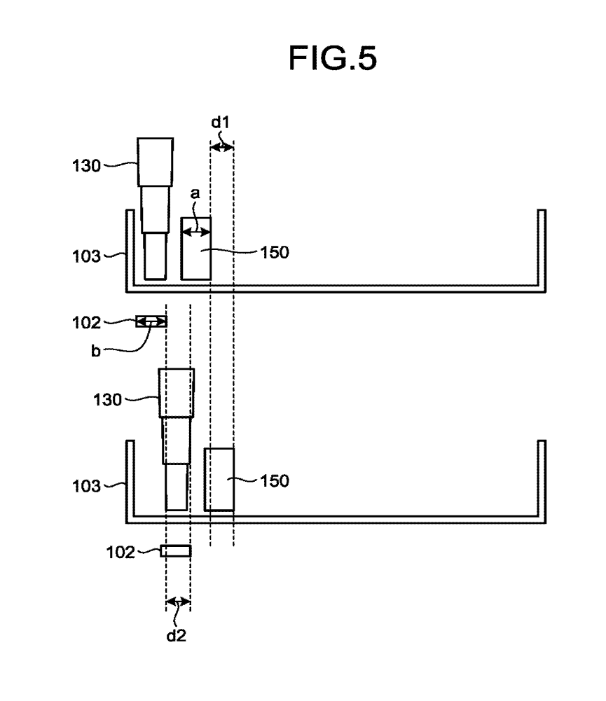

To avoid this situation, the mammography apparatus 1 according to the first embodiment is configured to exercise control so that no gap is formed between the image acquisition regions. More specifically, the operation controlling circuitry 111 exercises control so that the moving distance, at each time, of the X-ray detector 102 and the moving distance, at each time, of the ultrasound probe 150 are each equal to or shorter than the respective dimension thereof in the direction orthogonal to the depth direction of the compression plate 103. FIG. 5 is a drawing for explaining examples of the moving distances of the X-ray detector 102 and the ultrasound probe 150 according to the first embodiment. FIG. 5 illustrates an example in which the mammography apparatus 1 is viewed in the direction indicated by the arrow 12 in FIG. 2. For example, as illustrated in FIG. 5, the operation controlling circuitry 111 moves the ultrasound probe 150 in such a manner that a moving distance “d1” of the ultrasound probe 150 is shorter than the width “a” (the dimension in the widthwise direction) of the ultrasound probe 150. Further, as illustrated in FIG. 5, the operation controlling circuitry 111 moves the X-ray detector 102 in such a manner that a moving distance “d2” of the X-ray detector 102 is shorter than the width “b” (the dimension in the widthwise direction) of the X-ray detector 102. By moving these functional units in this manner, the X-ray images and the ultrasound images each include an overlapping region between any two pieces of data positioned adjacent to each other. It is therefore possible to acquire the data of the entire imaged object.

As explained above, the mammography apparatus 1 according to the first embodiment is configured to acquire an ultrasound image before acquiring an X-ray image. Accordingly, the mammography apparatus 1 is also capable of controlling the X-ray image acquiring process on the basis of information from the ultrasound image acquired prior. For example, the controlling function 241 is able to estimate a composition of the imaged object on the basis of brightness values of the ultrasound image and to determine an X-ray radiation condition in accordance with the estimated composition. Generally speaking, regarding X-ray image acquiring processes, in a comparison between mammary glands and fat, mammary glands attenuate X-rays more than fat does. Consequently, when a region having many mammary glands is to be imaged, it is desirable to increase the X-ray dose, compared to the situation where a fat region is to be imaged.

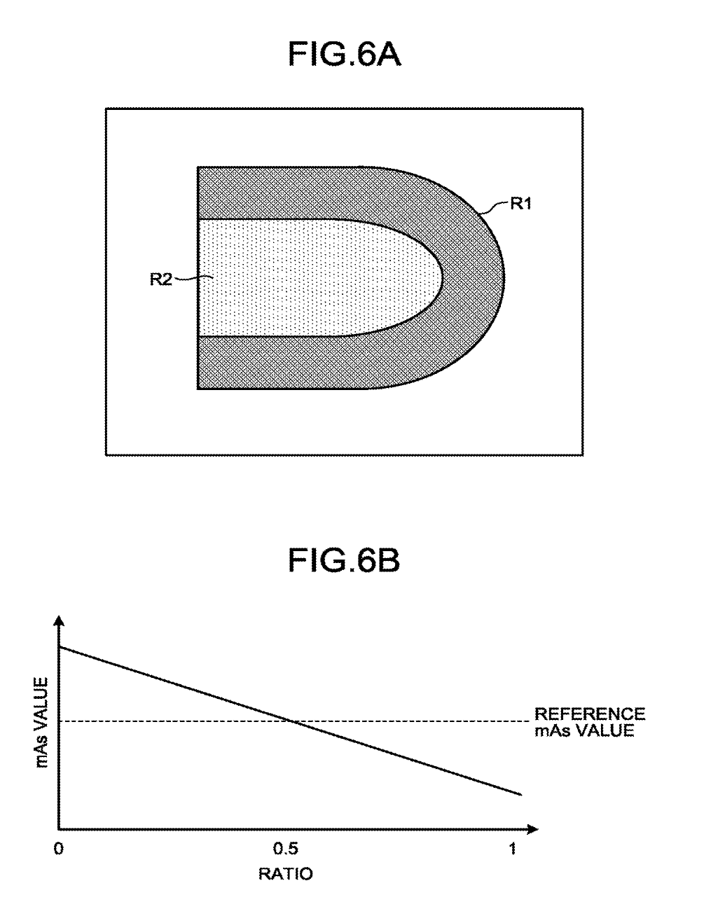

Accordingly, for example, the controlling function 241 is configured to calculate a ratio between mammary glands and fat for each region, on the basis of the brightness values of an ultrasound image and to further determine a radiation condition for each region on the basis of the calculated ratio. FIG. 6A is a drawing illustrating an example of the ultrasound image according to the first embodiment. As illustrated in FIG. 6A, in ultrasound images, a fat region is rendered with darker gray as indicated in a region R1, whereas a mammary gland region is rendered with lighter gray as indicated in a region R2. Consequently, for example, the controlling function 241 is configured to modulate an X-ray tube current in accordance with a ratio between brightness values.

FIG. 6B is a chart illustrating an example of an X-ray radiation condition according to the first embodiment. FIG. 6B illustrates a radiation condition where the horizontal axis expresses a ratio between pixel values (the darker gray/the lighter gray), whereas the vertical axis expresses an X-ray tube current value as a “mAs value”. For example, as illustrated in FIG. 6B, the radiation condition is set in such a manner that the lower the ratio (the darker gray/the lighter gray) is, the larger is the X-ray tube current value and that the higher the ratio (the darker gray/the lighter gray) is, the smaller is the X-ray tube current value. In other words, the radiation condition is set so that the X-ray tube current value is increased as the ratio of the mammary glands becomes higher, and conversely, the X-ray tube current value is decreased as the ratio of the fat becomes higher. The storage 230 is configured to store therein a radiation condition such as that illustrated in FIG. 6B for each of different values of breast thickness. The controlling function 241 is configured to calculate a ratio between pixel values for each of the regions from which an X-ray image is to be acquired, on the basis of the ultrasound image acquired prior. Further, the controlling function 241 is configured to read a radiation condition corresponding to the breast thickness of the patient and to determine an X-ray tube current value for each of the regions on the basis of the calculated ratio.

In this situation, as explained above, when the X-ray radiation condition is varied for each of the regions, the brightness values in the X-ray image are based on the radiation condition that is different for each region. Accordingly, for the purpose of arranging the X-ray image to appear as if the entire X-ray image were acquired under a constant radiation condition, the X-ray image generating function 243 multiplies the brightness values in each of the regions with the ratio in the radiation condition. As a result, it is possible to acquire the X-ray image with an appropriate amount of radiation exposure.

In the example described above, the example is explained in which the radiation condition is varied for each of the regions; however, possible embodiments are not limited to this example. It is possible to vary the radiation condition in an arbitrary manner. For example, it is acceptable to continuously vary the X-ray tube current value, on the basis of continuous changes in the ratio between the brightness values in the ultrasound image.

As explained above, the mammography apparatus 1 according to the first embodiment is capable of varying the X-ray radiation condition, on the basis of the ultrasound image acquired prior. Further, the mammography apparatus 1 is also capable of controlling the radiating and the stopping of the X-rays, on the basis of the ultrasound image acquired prior. More specifically, the controlling function 241 is configured to assess the position of the breast of the patient on the basis of the ultrasound image and to control the radiating and the stopping of the X-rays on the basis of the assessed position.

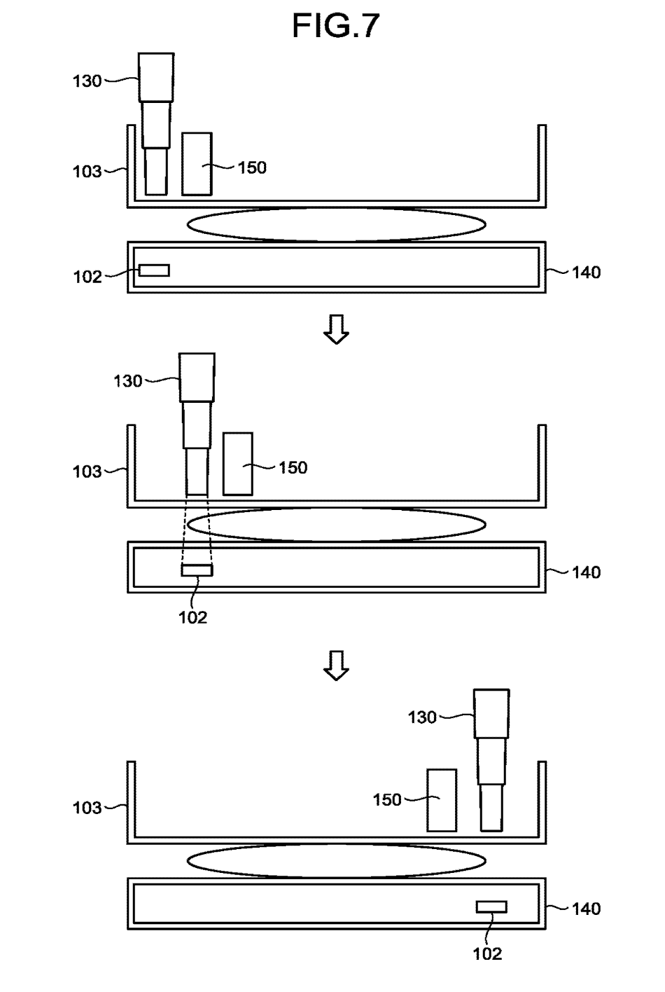

FIG. 7 is a drawing illustrating an example of the X-ray radiation control according to the first embodiment. FIG. 7 illustrates an example in which the mammography apparatus 1 is viewed in the direction indicated by the arrow 12 in FIG. 2. For example, as illustrated in the top section of FIG. 7, after an ultrasound image acquiring process is started, the controlling function 241 moves the X-ray detector 102 and the tubus 130 until the X-ray detector 102 and the tubus 130 reach the position where the breast is rendered in the acquired ultrasound image, while no X-rays are being radiated from the X-ray tube 101. After that, when the X-ray radiation region has reached the position where the breast is rendered in the ultrasound image, the controlling function 241 causes X-rays to be radiated from the X-ray tube 101, as illustrated in the middle section of FIG. 7. Subsequently, when the X-ray radiation region has reached the position where the breast is not rendered in the ultrasound image, the controlling function 241 stops the X-ray radiation from the X-ray tube 101, as illustrated in the bottom section of FIG. 7.

As explained above, the mammography apparatus 1 according to the first embodiment is configured to acquire an ultrasound image before acquiring an X-ray image. With this arrangement, the mammography apparatus 1 is able to acquire both of the two types of images of the breast having the same shape. It is therefore possible to improve the efficiency in image interpretation. Further, the mammography apparatus 1 is capable of acquiring the ultrasound images and the X-ray images at the same time. It is therefore possible to reduce pains of the patients taking breast cancer examinations (by exposing their breasts twice) and to shorten the time period required by the examinations.

Further, after having acquired the ultrasound images and the X-ray images, the mammography apparatus 1 causes the display 220 to display the acquired two types of images. For example, the display controlling function 244 is configured to cause the display 220 to display the acquired ultrasound and X-ray images so as to be positioned parallel to each other. Further, because the mammography apparatus 1 is configured to acquire the ultrasound images and the X-ray images by using mutually-the-same coordinate system, it is possible to easily align the positions of the two types of images. Accordingly, for example, the display controlling function 244 is configured to cause the display 220 to display the acquired ultrasound and X-ray images in a superimposed manner. In that situation, for example, the display controlling function 244 arranges the X-ray image to be displayed by using a gray scale, while arranging the ultrasound image to be displayed by using a color scale.

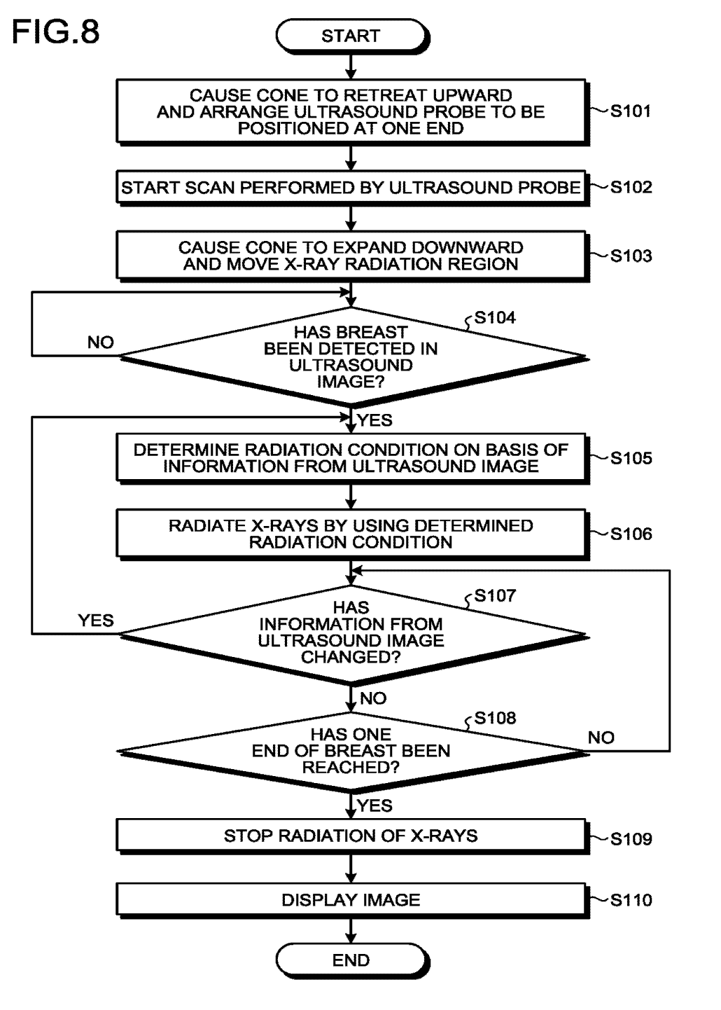

Next, a procedure in a process performed by the mammography apparatus 1 according to the first embodiment will be explained. FIG. 8 is a flowchart illustrating the procedure in the process performed by the mammography apparatus 1 according to the first embodiment. For example, the processes at steps S104 through S109 illustrated in FIG. 8 are realized as a result of the processing circuitry 240 invoking and executing the program corresponding to the controlling function 241 from the storage 230. Further, for example, the process at step S110 is realized as a result of the processing circuitry 240 invoking and executing the program corresponding to the display controlling function 244 from the storage 230.

At step S101, the operation controlling circuitry 111 causes the tubus 130 to retreat upward and arranges the ultrasound probe 150 to be positioned at one of the ends. At step S102, the transmitting and receiving circuitry 112 starts a scan performed by the ultrasound probe 150. At step S103, the operation controlling circuitry 111 causes the tubus 130 to expand downward and moves the X-ray radiation region. At step S104, the processing circuitry 240 judges whether or not the breast has been detected in the ultrasound image.

In this situation, when the breast has been detected in the ultrasound image (step S104: Yes), the process proceeds to step S105 where the processing circuitry 240 determines a radiation condition on the basis of information from the ultrasound image (e.g., a ratio between brightness levels). In this situation, the processing circuitry 240 continues to perform the judging process unless the breast is detected in the ultrasound image (step S104: No). After that, at step S106, the processing circuitry 240 exercises control so that X-rays are radiated under the determined radiation condition.

At step S107, the processing circuitry 240 judges whether or not the information from the ultrasound image (e.g., the ratio between the brightness levels) has changed. For example, the processing circuitry 240 judges whether or not the ratio between the brightness levels in the ultrasound image for each of the X-ray radiation regions has changed from a ratio between the brightness levels in the immediately preceding region. When the information from the ultrasound image has changed (step S107: Yes), the processing circuitry 240 returns to step S105 where the processing circuitry 240 determines a radiation condition.

On the contrary, when the information from the ultrasound image has not changed (step S107: No), the processing circuitry 240 proceeds to step S108 where the processing circuitry 240 judges whether or not an end of the breast has been reached in the ultrasound image. When an end of the breast has been reached (step S108: Yes), the processing circuitry 240 proceeds to step S109 where the processing circuitry 240 stops the radiating of the X-rays. When an end of the breast has not yet been reached (step S108: No), the processing circuitry 240 continues to perform the judging processes at steps S107 and S108. When the radiating of the X-rays is stopped at step S109, the process proceeds to step S110 where the processing circuitry 240 causes the display 220 to display the ultrasound images and the X-ray images.

As explained above, according to the first embodiment, the compression plate 103 is configured to compress the breast of the patient. The X-ray tube 101 and the tubus 130 are configured to radiate X-rays while moving the radiation region of the X-rays along the direction orthogonal to the depth direction of the compression plate 103. The X-ray detector 102 is configured to move along the direction orthogonal to the depth direction of the compression plate 103, in conjunction with the moving of the X-ray radiation region realized by the X-ray tube 101 and the tubus 130. The ultrasound probe 150 is configured to transmit and receive the ultrasound wave while being moved along the direction orthogonal to the depth direction of the compression plate 103. Consequently, the mammography apparatus 1 according to the first embodiment is able to acquire the two types of images of the breast having mutually the same shape. It is therefore possible to improve the efficiency in image interpretation.

Further, according to the first embodiment, the X-ray tube 101, the tubus 130, and the X-ray detector 102 are structured in such a manner that the radiation axis of the X-rays goes through the center of the X-ray detector 102. Consequently, the mammography apparatus 1 according to the first embodiment is able to accurately acquire the X-ray images while moving.

Further, according to the first embodiment, the moving distance, at each time, of the X-ray detector 102 and the moving distance, at each time, of the ultrasound probe 150 are each arranged to be equal to or shorter than the respective dimension thereof in the direction orthogonal to the depth direction of the compression plate 103. Consequently, the mammography apparatus 1 according to the first embodiment is able to acquire the images of the entire imaged subject.

Further, according to the first embodiment, the ultrasound probe 150 is configured to transmit and receive the ultrasound wave to and from the breast of the patient, prior to the radiating of the X-rays onto the breast of the patient performed by the X-ray tube 101 and the tubus 130. Consequently, the mammography apparatus 1 according to the first embodiment is able to control the X-rays by using the ultrasound image acquired prior.

Further, according to the first embodiment, the controlling function 241 is configured to vary the X-ray radiation condition, on the basis of the ultrasound image generated on the basis of the reflected-waves received by the ultrasound probe 150. Consequently, the mammography apparatus 1 according to the first embodiment is able to acquire the X-rays image under the radiation condition suitable for the imaged object.

Further, according to the first embodiment, the controlling function 241 is configured to assess the position of the breast of the patient on the basis of the ultrasound image and to control the radiating and the stopping of the X-rays on the basis of the assessed position. Consequently, the mammography apparatus 1 according to the first embodiment is able to reduce unnecessary radiation exposures.

### Second Embodiment

Although the first embodiment has thus been explained, the present disclosure may be carried out in various different modes other than those explained in the first embodiment.

In the first embodiment described above, the example is explained in which an ultrasound image is acquired before an X-ray image is acquired; however, possible embodiments are not limited to this example. For instance, an X-ray image may be acquired first.

Further, in the first embodiment, the example is explained in which the X-ray tube current value is adjusted on the basis of the ratio between the brightness values in the ultrasound image; however, possible embodiments are not limited to this example. It is acceptable to adjust any other arbitrary conditions. For instance, an X-ray tube voltage may be adjusted.

Further, in the first embodiment, the example is explained in which the radiation condition is varied on the basis of the ratio between the brightness values in the ultrasound image; however, possible embodiments are not limited to this example. It is acceptable to use any other arbitrary type of information from the ultrasound image. For instance, it is acceptable to use an average value of brightness values in each of the regions in the ultrasound image.

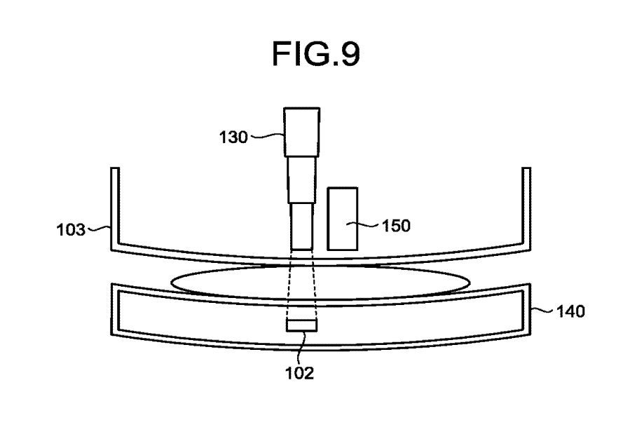

Further, the mammography apparatus 1 described in the first embodiment is merely an example. The structure of the apparatus may arbitrarily be configured. For instance, in the first embodiment, the example is explained in which the surfaces of the compression plate 103 and the Bucky's device 140 extending in the horizontal direction are each configured as a flat plane (see FIG. 7). However, possible embodiments are not limited to this example. For instance, the surfaces of the compression plate 103 and the Bucky's device 140 extending in the horizontal direction may each be configured as a curved plane. FIG. 9 is a drawing illustrating examples of the compression plate 103 and the Bucky's device 140 according to a second embodiment. FIG. 9 illustrates an example in which the mammography apparatus 1 is viewed in the direction indicated by the arrow 12 in FIG. 2. For example, as illustrated in FIG. 9, the compression plate 103 and the Bucky's device 140 according to the second embodiment may each be configured to have an arc shape curving toward the tubus.

As explained above, the mammography apparatus 1 is configured to vary the radiation direction of the X-rays along the left-and-right direction, as a result of the supporting unit 104 rotating while using the connection part of the X-ray tube 101 as a point of support, the supporting unit 104 being configured to support the X-ray tube 101. In other words, the mammography apparatus 1 is configured to vary the radiation direction of the X-rays with the arc-like movement by which the tip end side of the tubus 130 is swung in the left-and-right direction. Accordingly, by configuring the compression plate 103 and the Bucky's device 140 to each have an arc shape as illustrated in FIG. 9, it is possible to keep constant the distance between the breast compressed between the compression plate 103 and the Bucky's device 140 and the tip end of the tubus 130. It is therefore possible to acquire X-ray images having high quality.

Further, FIG. 9 illustrates the example in which the surfaces of both the compression plate 103 and the Bucky's device 140 extending in the horizontal direction are configured to have an arc shape; however, possible embodiments are not limited to this example. For instance, it is also acceptable to configure only the compression plate 103 to have an arc shape.

Further, in the embodiments described above, the example is explained in which the X-ray detector 102 moves along the direction orthogonal to the depth direction of the compression plate, in conjunction with the moving of the radiation region of the X-rays; however, possible embodiments are not limited to this example. The X-ray detector 102 does not necessarily have to move. In that situation, for example, the Bucky's device 140 has installed therein an area detector of such a size that is capable of detecting the X-rays of which the radiation direction is varied along the left-and-right direction.

Further, in the embodiments described above, the example is explained in which the mammography apparatus 1 is configured to determine the radiation condition for the X-rays on the basis of the ultrasound images, the mammography apparatus 1 being configured to acquire the ultrasound images and the X-ray images while moving the ultrasound probe 150 and the tubus 130; however, possible embodiments are not limited to this example. For instance, the mammography apparatus 1 may be configured to determine the X-ray radiation condition on the basis of an ultrasound image when acquiring the ultrasound image and an X-ray image of a region of interest (i.e., when acquiring the ultrasound image and an X-ray image of only a single region). In that situation, for example, the ultrasound image of the region of interest is acquired first. The controlling function 241 is configured to determine the X-ray radiation condition used for acquiring the X-ray image of the region of interest, on the basis of the acquired ultrasound image.

By using the medical image diagnostic apparatus according to at least one of the embodiments described above, it is possible to improve the efficiency in image interpretation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising:
a compression plate configured to compress a breast of a subject;
an X-ray tube configured to radiate X-rays;
an arm configured to hold the X-ray tube and to move, while the X-ray tube radiating the X-rays, an irradiation region of the X-rays in a direction perpendicular to a front-rear direction of the compression plate;
an X-ray detector configured to detect the X-rays having passed through the breast of the subject; and
an ultrasound probe configured to transmit and receive an ultrasound wave, the ultrasound probe being movable in the direction perpendicular to the front-rear direction of the compression plate.

2. The medical image diagnostic apparatus according to claim 1, wherein
the X-ray detector is configured to be moved in the direction perpendicular to a front-rear direction of the compression plate in conjunction with the movement of the irradiation region of the X-rays.

3. The medical image diagnostic apparatus according to claim 1, wherein
the X-ray detector is a photon-counting detector.

4. The medical image diagnostic apparatus according to claim 1, further comprising:
a collimator configured to adjust the irradiation region of the X-rays according to a position of the X-ray detector.

5. The medical image diagnostic apparatus according to claim 2, wherein
the irradiation region of the X-rays is moved by moving the arm holding the X-ray tube in conjunction with the movement of the X-ray detector.

6. The medical image diagnostic apparatus according to claim 2, wherein
the X-ray detector is configured to be moved by a distance equal to or less than the width of the X-ray detector in the direction perpendicular to a front-rear direction of the compression plate,
the ultrasound probe is configured to be moved by a distance equal to or less than the width of the ultrasound probe in the direction perpendicular to a front-rear direction of the compression plate.

7. The medical image diagnostic apparatus according to claim 1, wherein
the ultrasound probe is configured to transmit an ultrasound wave to the breast of the subject and to receive a reflective wave from the breast of the subject, in advance of the X-ray radiation to the breast of the subject.

8. The medical image diagnostic apparatus according to claim 1, further comprising:
processing circuitry configured to modify a irradiation condition of the X-rays based on a ultrasound image generated from a reflective wave received by the ultrasound probe.

9. The medical image diagnostic apparatus according to claim 8, wherein
the processing circuitry is configured to identify a position of the breast of the subject using the ultrasound image and to control start and stop of the X-ray radiation based on the identified position of the breast of the subject.

10. The medical image diagnostic apparatus according to claim 2, wherein
the X-ray tube and the X-ray detector are arranged so that a irradiation axis of the X-ray passes through a center of the X-ray detector.

11. The medical image diagnostic apparatus according to claim 1, wherein
the irradiation region of the X-rays and the ultrasound probe are configured to be moved outward from a central axis of the subject.

12. A medical image diagnostic apparatus comprising:
a compression plate configured to compress a breast of a subject;
an X-ray tube configured to radiate X-rays;
an X-ray detector configured to detect the X-rays having passed through the breast of the subject;
an ultrasound probe configured to transmit and receive an ultrasound wave; and
processing circuitry configured to modify a irradiation condition of the X-rays based on a ultrasound image generated from a reflective wave received by the ultrasound probe.
